(19) United States
(12) Patent Application Publication
(10) Pub. No.: US 2012/0107950 A1
(43) Pub. Date: May 3, 2012

(54) MEASUREMENT SYSTEM FOR FLUORESCENT DETECTION, AND METHOD THEREFOR

(76) Inventor: Bala S. Manian, Los Altos Hills, CA (US)

(21) Appl. No.: 13/034,515

(22) Filed: Feb. 24, 2011

(30) Foreign Application Priority Data
Nov. 3, 2010 (IB) PCT/IB10/54967

(51) Int. Cl. G01N 21/64; C12M 1/34

(52) U.S. Cl. 436/172; 422/82.08; 435/288.7

(57) ABSTRACT

In one aspect, the invention provides a measurement system, wherein the measurement system comprises a sample module to receive a sample, wherein the sample module comprises at least one fluorophore; an optics module to generate an incident beam to impinge on the sample to yield a laser spot; a detector module to detect fluorescence signals arising out of the sample; a processor module to process the fluorescence signals and provide relevant output; an output module; a control module to control the sample module, the optics module, the detector module and the output module. The measurement system of the invention is capable of measuring both bulk and event fluorescences of a sample containing at least one fluorophore. In another aspect, the invention provides a method for testing a fluid based on the measurement system of the invention.

### CROSS REFERENCE TO RELATED APPLICATION

This application claims priority from International Application No. PCT/IB2010/054967, filed on Nov. 3, 2010.

### FIELD OF THE INVENTION

The invention relates generally to a fluorescent measurement system and more specifically to a fluorescent measurement system that comprises a detection device that is capable of simultaneous bulk and event fluorescence measurement.

### BACKGROUND OF THE INVENTION

Fluorescent measurement that are robust in their construction and at the same time, are easy to use by operators of varying skill and expertise are required for a variety of applications.

WO 2005054854(A1) teaches a method wherein the test sample is combined with a fluorescence labeled ligand to said biological substance and the change in the fluorescence polarization of said test sample produced is detected by binding of said fluorescence-labeled ligand to said biological substance and a transponder for the wireless transmission of batch-specific data and/or measured values. The implement is provided with a reading module for the wireless transmission of data and power to the test element and an evaluation unit for evaluating the data or measured values received by the transponder. However, in these devices, the sample must be provided in well-machined smooth-surfaced sample tubes as errors due to surface irregularities have to be necessarily avoided.

In EP 0987535(A2), two or more scanned images are generated based on fluorescence data from dyes that have overlapping spectra. The two scanned images are processed using a linear regression analysis among corresponding pixels in the scanned images near certain cells to characterize relative contents of two fluorescing dyes in a target cell. Target cells are identified from the scanned images using processing resources which identify a peak sample within a neighborhood, and compare the amplitude of the peak with the amplitude of pixels on the perimeter of the neighborhood. Upon identifying a target cell in this manner, data from the plurality of scanned images corresponding to the identified cell are saved for further analysis. In JP 7301628 (A), using an automated device and the subject method, a scanning image forming blood cell calculator, in which an untreated biological fluid sample is made to react with a binder labeled with a fluorescent material, is provided. The sample is scanned optically, and fluorescent excitation is recorded. A space filter with sufficient pinholes is selected so that capacity measurement type detection in all the fluorescent targets in the respective column type areas can be carried out at the same time. The sample preparation methods and the operation of devices described in these patents are quite laborious and intensive.

The system mentioned in WO 2005001431(A2) uses a portable unit with an array of tunable lasers to excite a sample under test with a narrow band light source used to excite fluorescence. The fluorescent response is detected with a broadband detector and digitized. The information is then sent through wireless means to a remote server where a database of appropriate signatures is used to determine the identity of the sample. The results are sent back to the portable unit or to a Personal Digital Assistant (PDA). WO 9725678(A1) illustrates a network system and analysis of microscope slides and specimens which were originally computer encoded from a microscope and viewing locations and events of interest on the slide, with such information being stored on a network file server. For enhanced analysis, the computer terminals have direct access to patient background information. Such systems are capable of a single type of analysis, and adapting them to other types of analysis is quite difficult.

U.S. Pat. No. 7,270,970 provide systems and methods that process patient data, particularly data from point of care diagnostic tests or assays, including immunoassays, electrocardiograms, X-rays and other such tests, and provide an indication of a medical condition or risk or absence thereof. U.S. Pat. No. 5,554,340 describes a system for assaying a fluid sample, typically employing a fluorescent tag, the system comprising a lens capable of focussing both excitation and fluorescent radiation, a fluid-flow conducting conduit being provided in the lens extending transversely of the optical axis of and through the focal region of the latter. Such systems suffer from the drawback of precise fabrication of parts and components, including disposable components, which renders the system and its operation quite expensive.

In U.S. Pat. No. 5,380,663 a system for rapid microbead calibration of a flow cytometer including a suspension of quantitative fluorescent microbead standards and analytical software. The software is used to take information on the microbead suspension from a flow cytometer and analyze data, smooth curves, calculate new parameters and notify of expiration of the system. In U.S. Pat. No. 5,324,635 an analyzer that has a reaction disk for holding a plurality of reaction containers and a fluorophotometer for measuring fluorescence stemming from solutions in the containers is described. U.S. Pat. No. 5,093,271 talks about a method for the quantitative determination of antigen (or antibody) which comprises adding a sample containing an antigen (or antibody) to a dispersion of an insoluble carrier of fine particle size with an antibody (or antigen) fixed thereto to effect an antigen-antibody reaction, measuring absorbance of the reaction mixture at two different wavelengths and calculating the concentration of said antigen (or antibody) in the sample from the absorbance ratio. These systems are also limited in their applicability.

Hence, there is a dire need for a versatile system that is capable of or at least adaptable to, performing multiple detection of sample fluorescence which can withstand extreme conditions, such as scant-resources, and still can offer cost-effective results for a user.

### SUMMARY OF THE INVENTION

In one aspect, the invention provides a measurement system, wherein the measurement system comprises a sample module to receive a sample, wherein the sample module comprises at least one fluorophore. The measurement system also comprises an optics module to generate an incident beam to impinge on the sample to yield a laser spot, wherein the optics module is capable of displacing the laser spot relative to the sample volume. The measurement system then comprises a detector module to detect fluorescence signals arising out of the sample. The measurement system also comprises a processor module to process the fluorescence signals and provide relevant output, which output may be provided on an output module that forms part of the measurement system of the invention. The measurement system further comprises a control module to control the sample module, the optics module, the detector module and the output module.

In another aspect, the invention provides a method for testing a fluid based on the measurement system of the invention. The method includes providing sample module comprising at least one fluorophore to which a fluid is added to provide a sample, and subjecting the sample to the measurement system of the invention.

### BRIEF DESCRIPTION OF THE FIGURES

These and other features, aspects, and advantages of the present invention will become better understood when the following detailed description is read with reference to the accompanying drawings in which like characters represent like parts throughout the drawings, wherein:

FIG. 1 is a block diagram representation of the measurement system of the invention;

FIG. 2 is a block diagram representation of exemplary components of the measurement system of the invention;

FIG. 3 shows a schematic of the control electronics of the fluorescence detection device according to various embodiments;

FIG. 4 a schematic of the details of the motion control mechanism and the data acquisition submodule of the control electronics; and

FIG. 5 shows a back view of the interior of the detection device in one exemplary embodiment of the invention.

### DETAILED DESCRIPTION OF THE INVENTION

As used herein and in the claims, the singular forms “a,” “an,” and “the” include the plural reference unless the context clearly indicates otherwise.

As used herein an onsite location means a location that is adjacent or co-located with a referenced location, component, installation or combination thereof. Connectivity to the components that are onsite may be through wired communication means such as Ethernet connection, a USB port, a mezzanine connector, serial ports, or may be wireless such as through WAN, WLAN, Bluetooth, infrared connection and the like or through phone network.

As used herein a remote location means a location that is situated remotely from a referenced location, component, installation or combinations thereof. Connectivity to the components that are remotely located may be through a wireless connection such as through WAN, WLAN, Bluetooth, Infrared connection and the like or through phone network.

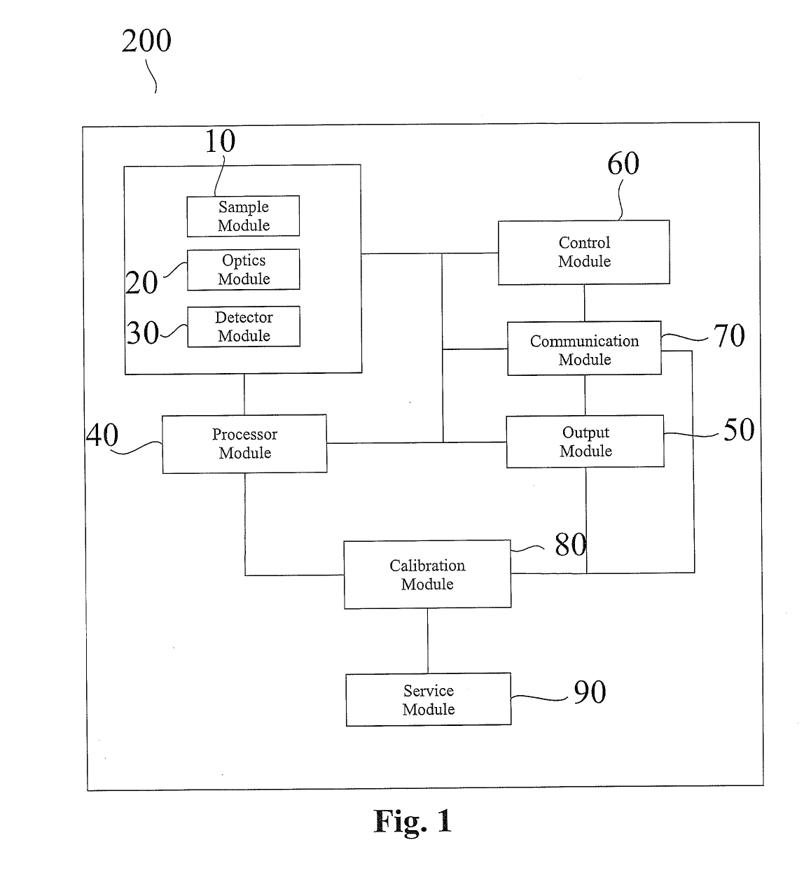

As noted herein, in one aspect, the invention provides a measurement system for sample, wherein the sample comprises at least one fluorophore. FIG. 1 shows a block diagram representation of the measurement system of the invention, wherein the measurement system is depicted by the numeral 200. The measurement system comprises a sample module 10 to receive a sample.

As used herein, sample means any substance that requires analysis for the purposes of either identification of one or more analytes, or measurement of properties, or quantification of one or more analytes, or the like, or combinations thereof. Sample may be in any given physical form, and this includes solution, suspension, emulsion, solid, and the like. In some embodiments, sample is an aqueous solution, and in other embodiments, sample is a suspension in an aqueous medium. Samples are typically derived from any number of sources. In one instance, sample is derived from a body fluid. Body fluids include saliva, sweat, urine, sputum, mucous, semen, and the like. In another instance, sample may be derived from a fluid source, such as water from a reservoir. In yet another instance, sample may be derived from a location such as a cotton swab of a baggage at security checkpoints, which may be used as such or may be suspended in a suitable solvent for analysis.

Samples useful in the invention comprise at least one fluorophore. Fluorophore as used herein means any moiety that is capable of being fluorescent upon excitation by a radiation corresponding to the excitation wavelength of the fluorophore, after which it emits radiation having a wavelength, which is referred to as emission wavelength. The fluorophore is attached to the remaining portion of the sample through physical linkages or through chemical linkages. Methods of incorporating fluorophores onto other materials are well-known to one of ordinary skill in the art, and can be arrived at without undue experimentation.

Sample is generally made available for the aforementioned purposes in a suitable sample carrier. Thus, the sample module of the invention includes a sample carrier as well. The nature of the sample carrier depends on the nature of the sample and analysis being performed. In some instances, sample carrier is a cuvette, in other instances, sample carrier is a well, in yet other instances, sample carrier is a plate, and in further instances, sample carrier is in the form of beads. The nature of the sample carrier will also accordingly determine the characteristics of the sample carrier. Thus, a cuvette is characterized by a wall thickness, a depth, a volume, and the like, while a well is characterized by a depth and a volume, and a plate is characterized by width. Sample may be pipetted into the sample carrier, or may be poured in, or may be added as a solid and spread along the surface through application of shear force, or prepared in situ in the sample carrier in a suitable medium, or through any other means known to those of ordinary skill in the art. In some instances, the fluorophore is part of the sample carrier, and the fluid to be analyzed is added to the sample carrier, and the sample comprising at least one fluorophore is prepared during the mixing.

The sample module 10 of the invention also includes a sample holder that comprises at least one receptacle. The receptacle is shaped to receive the sample carrier, and thus the receptacles and the sample holder may be shaped to facilitate receiving of the sample carrier.

The sample module 10 further comprises a movable platform that is attached to the sample holder. The movable platform and the sample holder are attached through any means known to those skilled in the art, and may include, for example, mechanical locking, magnetic locking, fasteners, screws, and the like. The movable platform is capable of moving in a linear trajectory, an arcuate trajectory, and combinations thereof. In one embodiment, the movable platform comprises a stepper motor, and the movement of the movable platform is effected by the suitable control of stepper motor. The sample, the sample carrier, the sample holder and the movable platform may sometimes be referred to as a sample assembly for purposes of this invention.

The sample module 10 may further comprise other additional functional units such as a vortex mixer to effect efficient mixing between an analyte and a fluorescent reagent to prepare a sample. Further, the sample module 10 may comprise an incubator capable of controlling temperature to effect efficient reaction, along with a timer to ensure complete reaction. Sample module 10 may also comprise a transferring means, such as a robotic arm, to transfer the sample or the sample carrier onto the sample assembly for measurement.

The measurement system also comprises an optics module 20, wherein the optics module 20 comprises a light source to generate an incident beam. The incident beam has a predefined wavelength that matches the excitation wavelength of the fluorophore in the sample. The incident beam also is characterized by a focus diameter. The incident beam is allowed to focus on the sample module to yield a laser spot to define a sample volume. The optics module 20 further comprises a displacing means to displace the laser spot relative to the sample volume in a depth dimensional space defined by the sample volume. The incident beam excites the at least one fluorophore in the sample volume to yield at least one emitted fluorescence signals. The region within the sample volume containing higher number of fluorophores, which will be evidenced by higher emitted fluorescence signals, is referred to as individual volume of interest. Then, the optics module 20 is used to focus the laser spot onto the at least one individual volume of interest to identify microvolumes of interest, from which concentrated emitted fluorescence signals emanate. The emitted fluorescence signals and the concentrated emitted fluorescence signals may be referred to as simply fluorescence signals in this invention. The optics module 20 may further comprise other lenses and filters to focus and improve quality of the laser spot incident on the sample, such that the laser spot has a suitable intensity and amplitude. The optics module 20 is capable of running multiple scans of the sample volume to ensure complete coverage of the sample in all three dimensions.

The measurement system of the invention comprises a detector module 30 to detect the at least one of one or more emitted fluorescence signals and at least one concentrated emitted fluorescence signal from the sample module. The detector module 30 comprises at least one beam splitter to split the fluorescent signals into at least two spectral bands. In one embodiment, the fluorescent signals are split into three spectral bands, the first spectral band has a wavelength that ranges from about 650 nm to about 690 nm, the second spectral band ranges from about 690 nm to about 740 nm, and the third spectral band ranges from about 740 nm to about 800 nm. The detector module 30 may further comprise filters to remove any unwanted and stray laser beams so as to detect only the fluorescent signals emanating from the sample. The detector module 30 may also comprise a reference point, which detects a reference beam to account for any variations or corrections, if necessary.

As is shown in FIG. 1, the sample 10, the optics module 20 and the detector module 30 may be present as a single unit. These modules may be combined with one or more other modules as described herein to form a device that forms part of the measurement system 200 of the invention. The appropriate combinations will become obvious to one of ordinary skill in the art. Further, in some embodiments all the modules may be located onsite and may also be a part of a single integral device. In some other embodiments only select modules may be located on site and others may be located remotely for better control, monitoring, maintenance purposes.

The measurement system of the invention also comprises a processor module 40 to process the one or more emitted fluorescence signals and at least one concentrated emitted fluorescence signal to provide a normalized bulk fluorescence reading and one or more event fluorescences for the fluid. The processor module 40 is capable of taking each individual fluorescence signals and treating them as a single data point, and at the same time is capable of stitching together each individual fluorescence signal and forming a data set that is useful for a variety of different interpretations by appropriate means. Alternately, the processor module 40 is capable of taking individual fluorescence signals and using it for further interpretations. Typical interpretations include identifying the presence or absence of a moiety being tested for, quantification of the concentration of a particular substance, and the like. Other interpretations also include verification of results using this system, wherein the results were obtained through other existing devices and systems.

The processor module 40 may also be capable of further processing of the bulk fluorescence and event fluorescence data. The further processing may include identifying a disease condition based on the data. The disease condition may then be classified into at least one of an onset, a progression, a regression, stable, an advanced condition. Such a disease condition may be arrived at based on other factors such as medical history, general well-being of a patient, patient's diet, age, weight and so on. Data representing the above-mentioned factors may be included as part of the processor module 40 using appropriate data storage means. The further processing may also include providing further treatment options that may comprise for example, providing action suggestions through the output module 50 to the user. Such action suggestions may include, for example, treatment methods. In a further embodiment, the further treatment options may also include taking appropriate actions, such as administering of pharmaceutical actives like insulin to a patient.

The measurement system of the invention then comprises an output module 50 to provide an output based on the normalized bulk fluorescence reading and one or more event fluorescences. Output may be in the form of graphical display, numerical displays especially for quantification measurements, color-coded displays wherein a color is used to represent a certain feature such as red for positive and green for negative for an analyte, simple monosyllable displays such as ‘Yes’ or ‘No’ to indicate the presence or absence of an analyte, and so on, and combinations thereof.

The measurement system of the invention also comprises a control module 60, which is used to control the sample module, optics module, the detector module and the output module. The speed of movement of the sample module 10 in a linear trajectory and an arcuate trajectory is defined by the nature of the analysis and is controlled by the control module. The nature of analysis may be chosen or predefined from a menu on a graphical user interface (GUI), and accordingly the speed of movement of the sample module is control module. The control module 60 is also used to control the optics module 20 to focus the laser spot onto the sample, and to also onto the microvolume of interest. The control module 60 is also used to displace the laser spot in a depth dimensional space across the sample. The displacement means may be optical means, or may be mechanical means of the movable platform as described hereinabove. The control module 60 is further used to control the detector module 30 to detect the fluorescence signals by activating the appropriate optics and electronics parts of the detector module 30.

As used herein, user means any person using the measurement system 200. In one exemplary embodiment, the user may be a technician who is assigned the task of measuring blood glucose levels, and in another exemplary embodiment, the user may be a doctor who is responsible for analyzing and interpreting the test results from the measurement system, and in yet another embodiment, the user may be a service technician who is responsible for the upkeep and maintenance of the measurement system.

The measurement system 200 also includes a communication module 70 to facilitate communication between the various modules and also to communicate with an appropriate user. The nature of communication may include, for example, communicating the input from a graphical user interface (GUI) to the controller module 60 for the operation conditions of the sample module 10 and the optics module 20, exporting results of the measurement to an output module 50, communicating the output from the output module to a user interface including a GUI or a printer, providing treatment options instructions to the user, and the like. The communication module 70 may also be used to communicate a status of the measurement system. The status of the measurement system may include the total number of different analyses the system has conducted, the total number of different analyses conducted on a given day, the various kinds of analyses performed, the number of times the light source has been turned on and off, the distance traversed by the sample module during the course of use, and the like. Such information will be useful to predict and/or schedule and conduct maintenance jobs on the system. According to various embodiments, communication may be effected through means known to those skilled in the art, and may include, for example, wired connection such as a Ethernet protocol, RS232, a parallel interface, a dedicated computer connection, wireless connection, Bluetooth, infrared, and the like. Typical operations of a communication module 70 may involve a communication protocol that is based on an algorithm written in an appropriate programming language. Further, depending on the capability of the communication module 70, the modules may be present as part of a single device, or some of the modules may be present remote to each other. For example, the optics module 10, sample module 20, the detector module 30 and the controller module 60 are present as part of one device, while the processing module 40 and the output module 50 are present remote to the device and these remote modules are connected to the device through a communication module 70. Other such combinations of the modules present adjacent to each other and remote to each other will become obvious to one of ordinary skill in the art.

The measurement system of the invention further comprises a calibration module 80 to monitor a status of the measurement system. The calibration may be effected by the inclusion of a calibration compound as part of the system, where the optics can be focused and accordingly tested. Suitable calibration compounds include Raman-active compounds, such as for example acetaminophen.

Thus, if the calibration module 80 indicates components in the optics module 20 are misaligned, or if the light source is losing power, then such messages may be transmitted through the communications module 70 to the output module 50. Depending on the nature of the message, an action may be performed to rectify any situations and enable smooth operation of the device. One exemplary action may include perform alignment of the optics module 20. Thus, the measurement system of the invention further comprises a service module 90 to indicate the need to perform service operations on the measurement system based on the status of the measurement system. Such indications may be, for example, providing color-coded icons representing the appropriate module in need of a service operation, or flashing indicators to give a visual indication for service need of the appropriate module, and the like.

Performance of service operations also include the replacing of at least one of the optics module 20, sample module 10, detector module 30, processing module 40, output module 50, control module 60, communication module 70 and combinations thereof.

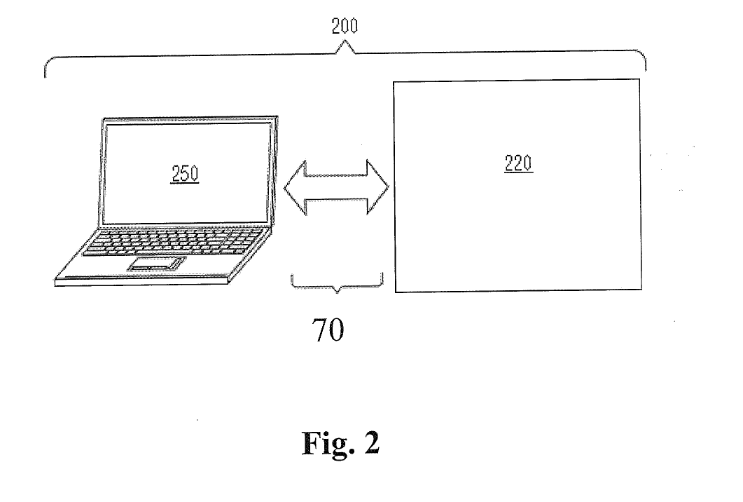

It will be obvious to one skilled in the art that a programmable analysis device can be used to operate and control the various modules of the invention. FIG. 2 shows a block diagrammatic representation of some of the components of the measurement system 200. It comprises a device 220, wherein said device comprises necessary components required to perform measurements. Such components may include, for example, the sample module 10, the optics module 20, and the detector module 30. The device 220 may further comprise the communication module 70, the processor module 40, and the like. The measurement system 220 also comprises a programmable analysis device 250, such as a laptop computer, a general purpose computer, a specific purpose computer, and the like. The programmable analysis device 250 can be programmed to control the operation of the device 220, to receive sample data transmitted from the device 220, and to analyze the sample data using computerized software algorithms. For example, the programmable analysis device 250 can be programmed to control the device 220 that comprises various modules as components of the device such that a specific scanning sequence is performed based on the nature of the sample module (not shown) to be loaded onto the device 220. The programmable analysis device 250 can be arranged to interface with the device 220 through the communication module 70.

As will be discussed below, each of the device 220 and the programmable analysis device 250 can include integrated hardware ports to allow sample image data and instructions to be transmitted therebetween.

The different embodiments of the measurement system as described herein allow for sample analysis to be conducted in a point-of-care method and the resulting data can be sent remotely to the programmable analysis device 250. Accordingly, the measurement system 200 can be used to readily analyze samples by medical authorities (such as physicians, nurses, clinicians, and the like) situated in the vicinity of the device 220 or where the programmable analysis device 250 is located.

Moreover, the programmable analysis device 250 can be arranged to be remotely operated, serviced, and/or supported by any relevant skilled personnel located anywhere in the world. As such, the device 220 and/or the programmable analysis device 250 can be provided with additional communication ports as needed to allow connection with a wide-area computer network. Moreover, the programmable analysis device 250 can be designed to allow an operator to forward the analyzed data to relevant skilled personnel or experts situated anywhere in the world.

As mentioned herein, the programmable analysis device 250 receives and/or extracts data from the device 220. The programmable analysis device 250 can be loaded with application-specific image processing software for receiving and extracting the sample data. Application-specific image processing software as used herein means the software is specific to an analysis protocol. For example, the nature of processing of data for an immunoassay may be different from the nature of processing of data for a flow cytometry assay. The image processing software can display operational information on the graphical user interface of the programmable analysis device 250 and can define the parameters of the scan to be conducted by the device 220. The image processing software allows the programmable analysis device 250 to receive and extract sample data measured by the device 220 and process it in a manner depending on the type of assay being conducted. While the programmable analysis device 250 and the device 220 are shown as separate devices in FIG. 2, they can be arranged in a single housing and/or integrated into a unitary device, as would be appreciated by one of ordinary skill in the art.

The application specific image processing software of the programmable analysis device 250 can correlate detected and extracted data with known data to produce analytical results. For example, the programmable analysis device 250 can receive sample data from the emitted signals associated with each fluorescently scanned particle of a fluid sample. A class of particles can be established based on the common characteristics of the class of particles. The data from a known class of particles can be compared to the data detected from sample particles of an unknown class. The processed data and interpreted results can be given as output to a user on the graphical user interface of the programmable analysis device 250.

The programmable analysis device 250 can include a central processing unit, a memory storage such as a hard drive, and a graphical user interface. The central processing unit and memory storage can collect, extract, and process the data. Suitable computer hardware, as implemented in the programmable analysis device 250 of the present teachings, would be appreciated by one of ordinary skill in the art.

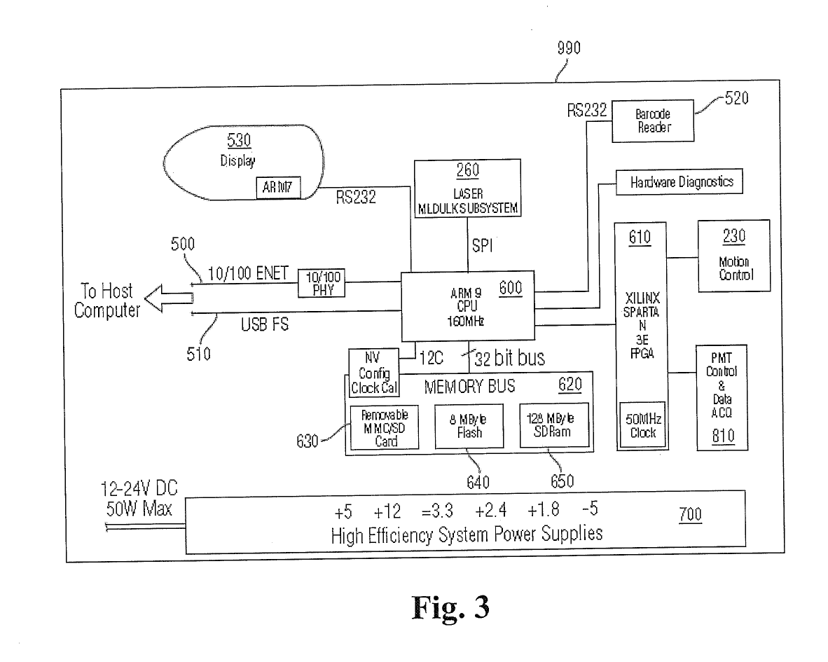

In an exemplary implementation of the measurement system of the invention, the control electronics of the fluorescence measurement device 220 is shown in FIG. 3. The main electronics board 990 can include the primary microprocessor (CPU) 600 and various user-accessible interfaces that can be used to control the measurement device 220. The primary microprocessor 600 can be, for example, an Atmel AT91SAM9260, which is an ARM9 microcontroller which can run at 160 MHz. As will be described below, a memory bus 620 can be provided which enables the use of large memories.

The primary microprocessor 600 can control the measurement sequence by commanding the hardware on the circuit board to turn motors by way of the motion control mechanism 230, to control the laser module subassembly 260, to read sensors, and to collect the sample image data. Volatile, programmable logic can be used to coordinate interrelated and time-critical tasks through the use of a field programmable gate array (FPGA) 610. The FPGA 610 can be a Xilinx Sparta N 3E running at 50 MHz.

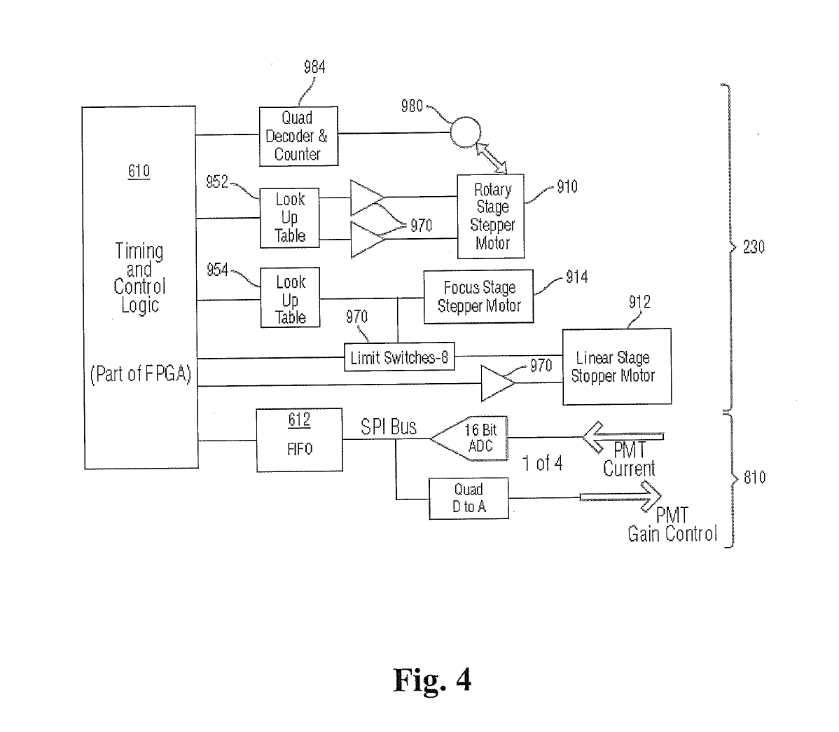

The FPGA 610 can also include embedded software for operation of a data acquisition submodule 810 and for operation of the motion control mechanism 230. The embedded software for the data acquisition submodule 810 can operate to control data acquisition from the PMTs 282, 284, 286 and to transmit the acquired data through the Ethernet port 500 to the programmable analysis device 250 at a fast rate. The embedded software for the motion control mechanism 230 can operate to control the motion of the sample holder 240 during the sample preparation sequence of the assay protocol, to control the motion of the components of the optics module as well as the sample assembly during data acquisition, and to establish a bi-directional interface through the programmable analysis device 250 to receive and set motion protocols and parameters. A more detailed discussion of the FPGA 610, and control of the data acquisition submodule 810 and the motion control mechanism 230 are discussed with respect to FIG. 4.

User-accessible interfaces on the measurement device 220 can include the communication port 500, one or more USB ports 510, a removable memory card 630 arranged on the memory bus 620, a barcode reader 520, a display 530, and others as needed. These and other user-accessible interfaces can be arranged on various portions of an exterior of the housing unit. Alternatively, a barcode reader can be incorporated for use with the programmable analysis device 250. During use of such a barcode reader, the data and image processing software of the programmable analysis device 250 could be programmed to require a scan of a pouch holding a sample to be analyzed and/or a container of application reagents to be used to prepare the sample to be analyzed to ensure that the proper accessories are matched with the desired scan being conducted by the measurement system of the invention 200. Moreover, the graphical user interface of the programmable analysis device 250 can be used as the display interface for the fluorescence measurement device 220. The graphical user interface of the programmable analysis device 250 can be used in lieu of, or in addition to, the display 530 which can be arranged on the fluorescence measurement device 220.

At least one power supply 700 can be arranged to supply the components of the fluorescence measurement device 220 with power from an external source. In an exemplary embodiment, the at least one power supply 700 can be arranged to supply varying voltages depending on the particular needs of different portions of the circuit board. In another exemplary embodiment, a 12V power source such as a battery may be used to supply 2.8 A power. Thus, the fluorescence measurement device 220 can consume relatively low power and could be operated through the use of a battery back-up system. Since the power requirements are quite low, several other sources of power can be contemplated for the operation of the measurement system 200. Such sources of power will be obvious to one of ordinary skill in the art.

Referring again to FIG. 3, the memory bus 620 can include a removable memory card 630, such as an SD card 630. The removable memory card 630 can be used to upgrade new software into the detection platform device 200. The memory bus 620 can also include a Flash memory card 640, such as an 8 MB flash memory card. The Flash memory card 640 can be used to store the program memory that runs the fluorescence measurement device 220. The memory bus 620 can also include a memory card 650, such as a 128 MB SDRAM memory card. The memory card 650 can be used to store the data as a scan takes place.

In one implementation, when the measurement device 220 is turned on, the microprocessor in this exemplary embodiment 600 is programmed to read the program saved on the Flash memory card 640 and is ready to execute instructions that can come into the microprocessor 600. Instructions come into the microprocessor 600 from the programmable analysis device 250 by way of the communication port 500. The microprocessor 600 then interprets the instructions and passes instructions to the FPGA 610. The FPGA 610 operates to coordinate all activities that need to be done for conducting a scanning sequence, for example, the FPGA 610 determines where to start scanning, how fast to rotate the sample assembly, how many data points to take, how far linearly to move the sample assembly, as well as other activities.

On the FPGA 610, there can be arranged a memory (FIFO 612, as will be discussed below) that can act as a buffer for data obtained during a scan which is continuously read by the microprocessor 600 and saved on the memory card 650 of the memory bus 620. A complete scan can be designed to take up a certain maximum amount of memory, such as, for example, 128 MB. When an entire scan is complete, it can be stored on the 128 MB SDRAM of the memory card 650. After the scan is completed, the microprocessor 600 communicates to the measurement device 220 that it has obtained the sample image data and can query when it will be ready to receive it. When the measurement device 220 indicates it is ready, the microprocessor 600 sends the data from the memory card 650 through the Ethernet port 500 to the programmable analysis device 250.

When the programmable analysis device 250 receives the data, the application-specific image processing software can review the sample image data, and by knowing what instructions it gave and the type of image data it has received, image data can be processed to produce a final sample data image for the user. For example, at the end of the scanning sequence, the application-specific image processing software can stitch together all the rotational passes that were made over the fluid sample to produce the final sample data image. The final sample data image can be displayed on the graphical user interface or can be provided to the user in any manner.

FIG. 4 shows the timing and control details of the FPGA 610 with regard to the data acquisition submodule 810 and the motion control mechanism 230. During a scan, data from all PMTs can be simultaneously sampled and converted.

FIG. 4 also schematically shows how data can be acquired from and how the gain can be adjusted for just one of the PMTs. However, the measurement device 220 can be arranged to include any number of PMTs. During operation of the measurement device 220, an output from any of the PMTs is an analog current reading, in which the strength of the current reading is proportional to the strength of the emitted fluorescent signal. This analog current reading is then converted to a digital signal by way of an analog-to-digital converter. All converted digital signals from the PMTs can then be buffered into a memory, such as a 64 kb FIFO 612. As discussed above, periodically the microprocessor 600 can be programmed to receive the data readings from the FIFO 612 and can be arranged to store the data readings in the memory card 650, thereby periodically emptying out the FIFO 612.

Moreover, the gain of the PMTs can be controlled by changing the amount of bias on the PMTs in order to control data acquisition. This is achieved by the application of signals from the microprocessor 600 to a particular PMT. Digital signals from the microprocessor 600 are converted to analog signals by way of a digital-to-analog converter to effect the PMT gain.

In one example, the PMT high voltage supply, which controls the PMT gain, is individually adjustable by DACs 614 on the main electronics board 990. PMT output is conditioned and converted to a binary value that is buffered in the FPGA 610 before being transferred to the CPU 600.

In one exemplary embodiment, with respect to the motion control mechanism 230, the FPGA 610 can control a rotary stepper motor 910, a linear stage stepper motor 912, and a focus stage stepper motor 914. The rotary stepper motor 910 can be arranged to rotate the sample assembly at a constant rotational speed. The linear stage stepper motor 912 can be arranged to continuously move the rotating sample assembly linearly during a scanning sequence. The focus stage stepper motor 914 can be arranged to move a focal lens up or down to a particular position (similar to a microscope) before a scanning sequence is started, and to then hold that lens position during the scanning sequence.

The rotary stage stepper motor 910 in one non-limiting example can be a 50-pole stepper having 4 windings. The rotary stage stepper motor 910 can be designed to rotate the sample assembly at a relatively low speed, such as, for example, 10 rpm, while providing a high level of repeatability between adjacent scans. Such a low-speed is sometimes used to improve signal-to-noise ratios. Moreover, as opposed to known stepper motors, the rotary stage stepper motor 910 of the present teachings can be capable of continuously rotating at a constant speed without a stepped rotary motion which can result in data reading errors. In a typical stepper motor, discrete signals are directed to a driver, resulting in the stepped motion. To prevent such a stepped motion, a look-up table 952 can be provided for the rotary stage stepper motor 910 which is used to direct current values to the poles of the motor so that the rotary stage stepper motor 910 sees a uniform magnetic field resulting in the continuous rotary motion without any stepping.

According to the present teachings, an integrated, protected dual H-bridge with external components and logic can be implemented to regulate the current precisely to the stepper motors 910, 912, 914. In the design of the present teachings, no heat-sinking or active cooling is required at the expected ambient conditions and with loads of less than 1 A peak per coil. More particularly, as shown in FIG. 4, the look-up table 952 of the FPGA 610 can be connected to power drivers 970 which operate to amplify the current values after they have been converted from digital to analog signals in the digital-to-analog converters. Since there are multiple windings going into the motor 910, each winding can be provided with a power driver 970.

An encoder 980 can be connected to the rotary stage stepper motor 910. By using position data from the encoder 980, or the frequency of the encoder signal, the measurement device 220 can operate to keep track of the angular position of the rotary motor 910 and ensure that the rotary motor 910 is rotating at a constant velocity. In addition, the encoder position can also be used to monitor the motor position during starting and stopping conditions.

As shown in FIG. 4, the focus stage stepper motor 914 can also be controlled through a look-up table 954. The focus stage stepper motor 914 can operate to adjust the focal lens to compensate for fabrication imperfections in the sample holders and/or sample carriers, to compensate for any misalignment, tilt, and/or wobble in the sample assembly, and any other inevitable misalignments. Since providing sample carriers that are perfectly flat is difficult and render the sample carriers very expensive, it is possible to provide compensation for any imperfections when conducting a rotary scan using the measurement device 220 that is part of the measurement system 200.

Such compensation can be achieved by taking a plurality of complete scans, each with the focal lens arranged at a different focal position, and then stitching the full images together by way of the application-specific image processing software. For example, a first scan can be conducted at a first focal position of lens, a second scan can be conducted by moving the focal position up a certain distance, such as, for example, 50 microns, and then a third scan can be conducted by moving the focal position down a certain distance, such as, for example, 50 microns. Afterwards, the application-specific image processing software can be used to process the three images together and create a theoretically perfect focal image since at least one of the scans should provide a region where the sample being scanned is in focus. In other words, the application-specific image processing software can be designed to take the sample data and process it to compensate for any variations in the focus.

As shown in FIG. 4, the linear stage stepper motor 912 and the focus stage stepper motor 914 can be controlled by photointerrupters 970. The measurement device 220 can include one or more photointerrupters 970 for limiting the motion of travel of the stages of the linear stepper motor 912. For example, one photointerrupter 970 can be arranged for a home position on each of the linear 912 and focus stages 914, and one for the sample carrier loading stage 912. Also, in a non-limiting example, each photointerrupter 970 is provided with a ground, 5V, a 499 ohm resistor pull-up to 5V for an LED, and an output signal. The photointerrupter 970 can be able to pull down a series 499 ohm resistor and 1.7V LED from 5V to within 0.4V of ground. The LED provides a visual indication of the state of the photointerrupter 970. For example, photointerrupters 1-6 can be routed to FPGA 610 inputs and can be read by the CPU 600 indirectly, while photointerrupters 5-8 can be directly connected to the CPU 600.

As disclosed above, the FPGA 610 can be arranged to coordinate the operation of the detection platform device 200. For example, the FPGA 610 can determine where to start scanning, how fast to rotate the sample assembly 240, how many data points to take, and how far to move the sample assembly 240 in a radial direction.

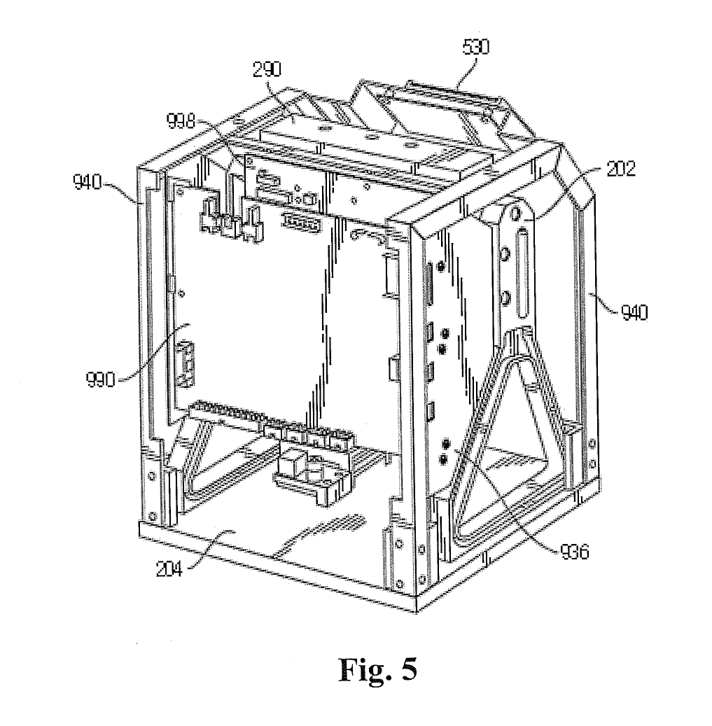

FIG. 5 shows one exemplary embodiment of the device 220, which comprises the main electronics board 990 secured vertically within the back portion of the measurement device 220. The main electronics board 990 can be arranged in parallel with the optics module 290 and secured by way of plates 936 to a frame 940 of the measurement device 220. The optics module 290 may be secured onto a circuit board 998. The LCD panel 530 is shown in the background and can also be secured to the frame 940. The entire assembly is held in place on a base platform 204, and is further supported by lateral support 202.

Thus, a robust measurement system for a fluorescent detection, analysis and quantification that is capable of obtaining both bulk fluorescent and event fluorescence measurements in harsh, resource-stressed environments, which consumes low power and so can be operated using any kind of power source is provided in the invention. This will enable facile use of such devices in a variety of situations, such as point-of-care diagnostics. The measurement system of the invention is also capable of being operated remotely. These advantages also make the measurement system of the invention most conducive for operations wherein some users and/or operators are present remote the device, such as telemedicine.

In another aspect, the invention provides a method testing a fluid sample. The method involves providing a sample reagent that comprises at least one fluorophore. Advantageously, the sample reagent may be dry to begin with so that transportation and storage of the sample reagent does not become an issue during regular use. The method then involves providing an analyte, such as, for example a body fluid. The analyte is then added to the sample reagent to prepare a sample. This sample is then added to the sample module that is present as part of the measurement system of the invention. Then, the sample is analyzed using the measurement system of the invention to provide results in a suitable manner for further use as described herein.

While only certain features of the invention have been illustrated and described herein, many modifications and changes will occur to those skilled in the art. It is, therefore, to be understood that the appended claims are intended to cover all such modifications and changes as fall within the true spirit of the invention.

I claim:

1. A measurement system comprising:
a sample module to receive a sample, wherein the sample module comprises at least one fluorophore, wherein the sample module is movable in a linear trajectory, an arcuate trajectory or combinations thereof;
an optics module comprising a light source to generate an incident beam having a predefined wavelength and a focus diameter onto the sample module to yield a laser spot that defines a sample volume, the optics module further comprising a displacing means to displace the laser spot relative to the sample volume in a depth dimensional space defined by the sample volume, wherein the sample volume comprises at least one individual volume of interest;
a detector module to detect at least one of one or more emitted fluorescence signals and at least one concentrated emitted fluorescence signal from the sample module;
a processor module to process the one or more emitted fluorescence signals and at least one concentrated emitted fluorescence signal to provide a normalized bulk fluorescence reading and one or more event fluorescences for the fluid;
an output module to provide an output based on at least one of the normalized bulk fluorescence reading and one or more event fluorescences; and
a control module, wherein the control module controls the sample module, optics module, the detector module and the output module.

2. The measurement system of claim 1 further comprising a communication module to communicate at least the output to a user interface or an input for the control module, or combination thereof.

3. The measurement system of claim 2 wherein the input is done from an onsite location or a remote location.

4. The measurement system of claim 1, wherein the output is at least one of a graphical representation, a numerical representation or a combination thereof.

5. The measurement system of claim 2, wherein the output is communicated to at least one of an onsite location or a remote location.

6. The measurement system of claim 2 further comprising a calibration module to monitor a status of the measurement system.

7. The measurement system of claim 6, wherein the communication module is used to communicate the status of the measurement system.

8. The measurement system of claim 7, wherein the status of the measurement system is communicated to an onsite location or a remote location.

9. The measurement system of claim 6 further comprising a service module to indicate a need to perform service operations on the measurement system based on the status of the measurement system.

10. The measurement system of claim 9, wherein the service operations include performing at least one of replacing the optics module, replacing the control module, replacing the detector module, replacing the output module, replacing the communication module and combination thereof.

11. The measurement system of claim 9, wherein the service module is located on an onsite location or a remote location.

12. The measurement system of claim 1, wherein the output module is situated remotely relative to the sample module, the optics module, and detector module.

13. A diagnostic assay system that uses the measurement system of claim 1.

14. An enzymatic assay system that uses the measurement system of claim 1.

15. An immunoassay system that uses the measurement system of claim 1.

16. A method for testing a fluid, the method comprising:
providing a sample reagent comprising at least one fluorophore;
providing an analyte for the sample reagent to prepare a sample;
providing a measurement system, wherein the measurement system comprises,
a sample module to receive a sample, wherein the sample module is movable in a linear trajectory, an arcuate trajectory or a combination thereof;
an optics module comprising a light source to generate an incident beam having a predefined wavelength and a focus diameter onto the sample module to yield a laser spot that defines a sample volume, the optics module further comprising a displacing means to displace the laser spot relative to the sample volume in a depth dimensional space defined by the sample volume, wherein the sample volume comprises at least one individual volume of interest,
a detector module to detect at least one of one or more emitted fluorescence signals and at least one concentrated emitted fluorescence signal from the sample module,
a processor module to process the one or more emitted fluorescence signals and at least one concentrated emitted fluorescence signal to provide a normalized bulk fluorescence reading and one or more event fluorescences for the fluid,
an output module to provide an output based on at least one of the normalized bulk fluorescence reading and one or more event fluorescences, and
a control module, wherein the control module controls the sample module, optics module, the detector module and the output module; and
obtaining a measurement for the sample based on the at least one fluorescent event and a bulk fluorescence reading.

17. The method of claim 16 further comprising communicating the measurement for the fluid to at least one of an onsite location or a remote location.

18. The method of claim 16 further comprising obtaining a status for the measurement system.

19. The method of claim 18 further comprising communicating the status for the measurement system to a service module.

20. The method of claim 19 further comprising servicing the measurement system using the status for the measurement system.

21. The method of claim 20, wherein servicing comprises performing at least one of replacing the optics module, replacing the control module, replacing the detector module, replacing the output module, replacing the communication module and combination thereof.
